(19) United States
(12) Patent Application Publication
(10) Pub. No.: US 2015/0098898 A1
(43) Pub. Date: Apr. 9, 2015

(54) PHARMACEUTICAL COMPOSITION, METHOD AND KIT FOR DETECTING HUMAN MELANOMA CELLS

(71) Applicant: Institute of Nuclear Energy Research Atomic Energy Council, Executive Yuan, Taoyuan County (TW)

(72) Inventors: Si-Yen LIU, Taoyuan County (TW); Sheng-Nan LO, Taoyuan County (TW); Wan-Chi LEE, Taoyuan County (TW); Wei-Chuan HSU, Taoyuan County (TW); Te-Wei LEE, Taipei City (TW)

(21) Appl. No.: 14/309,808

(22) Filed: Jun. 19, 2014

(30) Foreign Application Priority Data
Oct. 4, 2013 (TW) 102136034

(51) Int. Cl. A61K 51/12

(52) U.S. Cl. CPC A61K 51/1234
USPC 424/1.21

(57) ABSTRACT

The present disclosure is related to a pharmaceutical composition for detecting human melanoma cells, including: a liposome, a biomolecule having specificity for αᵥβ₃ integrin and a radionuclide. The present disclosure also provides a method using the pharmaceutical composition for detecting human melanoma cells and a kit performing the method. By means of the specificity of the biomolecule for the αᵥβ₃ integrin, the liposome is enabled to have recognition ability and facilitates the interaction between the liposome and a target cell. Therefore, the present disclosure is capable of being widely applied in the field of melanoma diagnosis, lymphatic metastasis detection and post-surgical monitoring, and so on.

### BACKGROUND

1. Technical Field

The present disclosure is related to a pharmaceutical composition, method and kit for detecting human melanoma cells, especially having specificity for αᵥβ₃ integrin expressed in melanoma cells.

2. Related Art

Liposome provides a variety of different directions for development of nanotechnology as a platform for research and development, and furthermore promotes applications of drug delivery in targeting tumor cells or tumor cell development. When the liposome reaches vessels near a tumor through blood circulation, by means of enhanced permeability and retention effects (EPR effects), liposome enters tumor tissues and is accumulated in the tumor. Through modification with polyethylene glycol, the stability of the liposome is improved and the circulation time of the liposome in the blood is increased. However, the interaction between the liposome and the target cells is reduced, thereby resulting in poor endocytosis and endosomal escape. Therefore, it attracts attentions that the polyethylene glycol-modified liposome is further designed and modified to be a targeting liposome. In addition to modification at the end with polyethylene glycol, a molecule having specificity ability can also be connected outside the liposome membrane.

αᵥβ₃ integrin is a cell adhesion receptor related to tumor angiogenesis and tumor metastasis. Since αᵥβ₃ integrin has high specific bonding ability with a peptide having a sequence of Arg-Gly-Asp (RGD), can promote cell attachment and cell growth, and is beneficial to wound healing. A radiolabled RGD peptide can be developed into an effective specific tumor imaging agent. It is reported documents that a variety of radionuclide labeled compounds have been used for detecting αᵥβ₃ integrin so far, so as to serve as a tumor imaging agent.

On the other hand, in clinical, Breslow thickness is used as the installment basis for melanoma, and in screening, a biopsy mainly serves as a basis for diagnosis. When the Breslow thickness of a patient with melanoma is less than or equal to 1 mm, generally, the tumor will be directly removed by a surgery, and the risk of lymphatic metastasis is very low (2% to 5%). When the Breslow thickness is greater than 2 mm, lymphatic metastasis will occur with the increase of the Breslow thickness, so it needs to determine whether lymphatic metastasis is possible by sentinel lymph node biopsy.

In the sentinel lymph node biopsy, a blue dye and a radioactive tracer (99mTc-sulfur colloid) are injected near the tumor tissue, a site with high radioactivity accumulation is determined as a sentinel lymph node by observing the blue dye with naked eyes or detecting with a detector, 1 to 5 sentinel lymph nodes are picked out by a surgery, and then whether cancer cells exist in the lymph node is pathologically interpreted. However, the lymphatic system is very complex, and due to high-density lymphatic basins, the problem of background interference signals occurs in use of the radioactive tracer, so the probability of identifying the sentinel lymph node is lowered. Furthermore, the use of the blue dye will last for several weeks, even some patients cannot discharge the blue dye, and imprinting-like marks can be observed with naked eyes.

Therefore, it is necessary to research and develop a novel reagent, method and kit for detecting melanoma cells.

### SUMMARY

In view of the disadvantages of the prior art, an objective of the present disclosure is to provide a pharmaceutical composition for detecting melanoma cells, comprising:

a liposome, having an outer membrane;

a biomolecule, connected to the outer membrane of the liposome, and having specificity for αᵥβ₃ integrin; and

a radionuclide, selected from the group consisting of indium, iodine, rhenium, gallium-67, gallium-68 and technetium.

In an embodiment, the liposome may be further modified by polyethylene glycol.

In an embodiment, the biomolecule may be a cyclic peptide. In a specific embodiment, the cyclic peptide may be cyclic RGDfK, but not limited thereto, and any biomolecule having specificity for αᵥβ₃ integrin and having fixed configuration can also be used.

In an embodiment, the radionuclide may preferably be indium-111, but not limited thereto.

An objective of the present disclosure is to provide a method for detecting human melanoma cells, comprising:

a. administering the pharmaceutical composition to a subject having human melanoma cells, wherein the human melanoma cells comprise αᵥβ₃ integrin; and

b. detecting data of specific binding of a biomolecule of the pharmaceutical composition and the αᵥβ₃ integrin, so as to detect the transfer degree of the melanoma cells.

In an embodiment, the melanoma cells may be A375.S2.

In an embodiment, the data of specific binding may be determined by using nano single ingle photon emission computed tomography (SPECT/CT) images.

In an embodiment, the subject may be an animal with xenotransplantation.

An objective of the present disclosure is to provide a kit for detecting human melanoma cells, comprising:

the above-mentioned pharmaceutical composition; and

an operating instruction, wherein the operating instruction comprises:

a. administering the pharmaceutical composition to a subject having human melanoma cells, wherein the human melanoma cells comprise αᵥβ₃ integrin; and

b. detecting data of specific binding of a biomolecule of the pharmaceutical composition and the αᵥβ₃ integrin, so as to detect the transfer degree of the melanoma cells.

In an embodiment, the melanoma cells may be A375.S2.

In an embodiment, the data of specific binding may be determined by using nano SPECT/CT images.

In an embodiment, the subject may be an animal with xenotransplantation, for example, a mouse.

### BRIEF DESCRIPTION OF THE DRAWINGS

The disclosure will become more fully understood from the detailed description given herein below for illustration only, and thus are not limitative of the disclosure, and wherein:

FIG. 1 shows an experimental procedure of Test Example 1 of the present disclosure;

FIG. 2 shows results of image acquisition at 24 h after injection of liposome to human melanoma tumor-bearing nude mouse in Test Example 1 of the present disclosure, where picture a shows an image after injection of ¹¹¹In-liposome; picture b shows an image after injection of ¹¹¹In-cyclic RGDfK-liposome; picture c shows an image after injection of ¹¹¹In-cyclic RGDfK-liposome and cyclic RGDfV peptide, in which the scale of ¹¹¹In in the pictures is 0 to 100% maximum signal strength and tumor slices 0 to 100% (the minimum to the maximum is 0 to 7.66×10e⁻⁵ dose);

FIG. 3 shows results of quantification of a region of interest (ROI) of a tumor tissue in Test Example 1 of the present disclosure;

FIG. 4 shows an experimental procedure of Test Example 2 of the present disclosure;

FIG. 5 shows results of image capturing at 24 h after injection of liposome to human melanoma tumor-bearing nude mouse in Test Example 2 of the present disclosure, where picture a shows an image after injection of ¹¹¹In-liposome; picture b shows an image after injection of ¹¹¹In-cyclic RGDfK-liposome, in which the scale bar for ¹¹¹In in FIG. 5 is 0 to 100% for maximum signal strength and tumor slices (minimum to maximum is 0 to 7.66×10e⁻⁵ injection dose);

FIG. 6 shows results of measurement of αᵥβ₃ integrin expression in Test Example 3 of the present disclosure, where the experimental results shown in FIG. 6 are representative examples in three independent experiments of three mice per group;

In FIGS. 7A AND 7B, figure A shows ratios of RAW 264.7 cells having optimal phagocytosis to fluorescence-labeled Escherichia coli determined in Test Example 4; figure B shows measurement results of influence of cyclic RGDK-liposome on phagocytosis of RAW 264.7 cells to fluorescence-labeled Escherichia coli, and picture a to picture c in figure B show the case of the presence of liposome; and picture d to picture f show the case of the presence of cyclic RGDfK-liposome.

FIG. 8 shows results of measurement of generation of reactive oxygen species by RAW 264.7 cells with liposome or cyclic RGDfK-liposome, where picture a shows the case of the presence of the liposome; and picture b shows the case of the presence of the cyclic RGDfK-liposome.

FIG. 9 shows data of influence of liposome or cyclic RGDfK-liposome on generation of reactive oxygen species by RAW 264.7 cells, where picture a shows data in the case of the presence of low-concentration liposome or cyclic RGDfK-liposome; and picture b shows data in the case of the presence of high-concentration liposome or cyclic RGDfK-liposome. All the experiments are performed 5 times.

### DETAILED DESCRIPTION

In the following, embodiments of the present disclosure are described in detail with the following examples, but not limited thereto. The foregoing and other objectives, features and advantages of the present disclosure will get clearer through the description below and the accompanying drawings.

### I Materials

(I) Cells

A375.S2 cells (Accession Number: BCRC 60263) and RAW 264.7 cells (Accession Number: BCRC 60001) were obtained from Bioresources Collection and Research Center. The A375.S2 cells were cultured in a minimum essential medium (MEM) containing 2 mML glutamine, 0.1 mM non-essential amino acids, 1 mM sodium pyruvate, 1.5 g/L sodium bicarbonate and 10% hot decomplemented fetal bovine serum decomplemented fetal bovine serum. The RAW 264.7 cells were cultured in a Dulbecco's modified Eagle's medium containing 4 mML-glutamine, 4.5 g/L glucose, 1.5 g/L sodium bicarbonate and 10% hot decomplemented fetal bovine serum. The cell culture environment was controlled to have a content of CO₂ of 5% and a temperature of 37° C.

(II) Reagents and Antibodies

Polyethylene glycol [PEG] ₂₀₀₀-carbamyl distearoylphosphatidyl ethanolamine (NHS-PEG-DSPE) was purchased from NOF Corporation, Japan. Cyclic RGDfK peptide was obtained from Peptides International, Inc. (Louisville, Ky.). FluoroProfie® protein quantification kit was obtained from Sigma (St. Louis, Mo.), and CytoSelect TM cell transformation assay kit was obtained from CELL BIOLABS, Inc. (San Diego, Calif.). Escherichia coli BioParticles®, Alexa Fluor® 594 conjugate and Escherichia coli BioParticles® opsonizing reagent were obtained from Invitrogen (Carlsbad, Calif.). Fluorescent isothiocyanate (FITC) labeled mouse anti-human CD51/CD61 monoclonal antibody and FITC-labeled mouse IgG1 isotype control monoclonal antibody were purchased from BD Biosciences (San Jose, Calif.).

### II Preparation Example

### (I) Preparation of Cyclic RGDfK-Liposome

6.72 mg NHS-PEG-DSPE and 1.2 mg cyclic RGDfK peptide (mole ratio 1.2:1) were dissolved in 1 mL dimethylformamide (DMF) and reacted for 24 h at room temperature, and the reaction solution was frozen with liquid nitrogen and the solvent DMF was removed by using a freeze dryer, the crystal after lyophilization was dissolved with dichloromethane, the solution was filtered by gravity filtration, and cyclic RGDfK-PEG-DSPE was remained on the filter paper. The filter paper was allowed to stand still till dichloromethane was completely volatilized, and then cyclic RGDfK-PEG-DSPE was dissolved with deionized water.

The theoretical molecular weight of cyclic RGDfK-PEG-DSPE was determined by using a matrix-assisted laser desorption/ionization time of flight mass spectrometer (MALDI-TOF/MS), where the acetonitrile-to-water ratio was 1:1 and 0.1% trifluoroacetate were used as the matrix solution, and 10 mg/ml α-cyano-4-hydroxycinnamic acid was loaded. In order to prepare cyclic RGDfK-liposome, 0.5 mg cyclic RGDfK-PEG-DSPE and 0.5 ml liposome solution were reacted for 30 min in a 60° C. water bath. The peptide insertion efficiency was determined according to the experimental plan provided by the manufacturer of the LuoroProfie® protein quantification kit.

### II) Preparation of ¹¹¹In-8-hydroxyquinoline (referred to as ¹¹¹In-oxine for shot hereafter

¹¹¹In having an activity of 2.18-3.1 mCi was added to 10 μL oxine (8-hydroxyquinoline, 8-hydroxyquinoline) (10 μg/μL absolute alcohol), the reaction volume was supplemented to 1 mL with 0.2 M sodium acetate buffer (pH 5.5), and the mixture was subjected to a reaction for 15 min at 50° C. The product ¹¹¹In-oxine was extracted with 1 mL chloroform, the organic layer was taken out and chloroform was drained off by using a rotary vacuum concentrator. ¹¹¹In-oxine was dissolved with 100 μL 20% absolute alcohol solution.

### (III) Preparation of ¹¹¹In-cyclic RGDfK-liposome

0.5 mg cyclic RGDfK-PEG-DSPE, 0.5 mL liposome and ¹¹¹In-oxine dissolved in 20% absolute alcohol were reacted for 30 min at 60° C., and the resulting product was purified with G-25 Sephadex gel.

### III Test Example

In test examples below, data was obtained from three independent experiments, and analysis was performed through Student's t test. The significance was set to be less than 0.05.

### (I) Test Example 1

### ¹¹¹In-Cyclic RGDfK-Liposome in Nano SPECT/CT®^{plus} Imaging and Quantitative Analysis of Images of Animal Model with Xenotransplantation of Human Melanoma Cells without Tumor Metastasis

1. Experimental Methods

Nano SPECT/CT/Micron Computed Tomography (MCT) Imaging

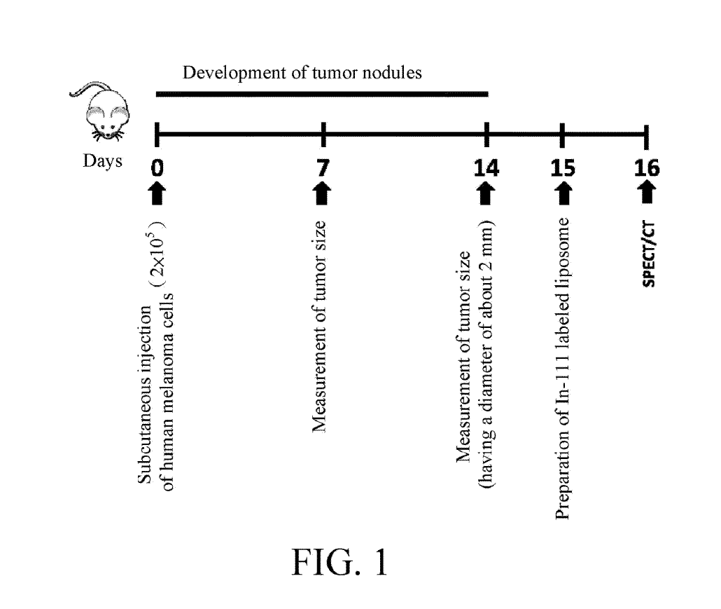

The animals used in this test example were female BALB/c AnN. Cg-Foxn1^{nu}/Cr1Nar1 mice, purchased from the National Laboratory Animal Center, Taiwan. The experimental plan had been approved by the Institutional Animal Care and Use Committee of the Nuclear Energy Institute. 2×10⁵ A375.S2 human melanoma cells were injected into a nude mouse at the neck. Two weeks after injection, the animal had developed a tumor having a diameter of about 2 mm. In order to determine the in vivo distribution of the radioactive liposome at 24 h after injection by using Nano SPECT/CT®^{plus}, first, the mouse was injected with ¹¹¹In labeled liposome (50 uCi) and anesthetized with 1.5% isoflurane at 24 h after injection, and nuclear images of the mouse were captured. For the experimental procedure, reference can be made to FIG. 1.

2. Experimental Results

This test example is to test in vivo target imaging data of cyclic RGDfK-liposome in animal model with xenotransplantation of human melanoma cells. Furthermore, in order to determine that tumor accumulation is caused by the cyclic RGDfK, the cyclic RGDfV peptide (1 mg/kg) that is known having strong binding with αᵥβ₃ integrin is administered to mice.

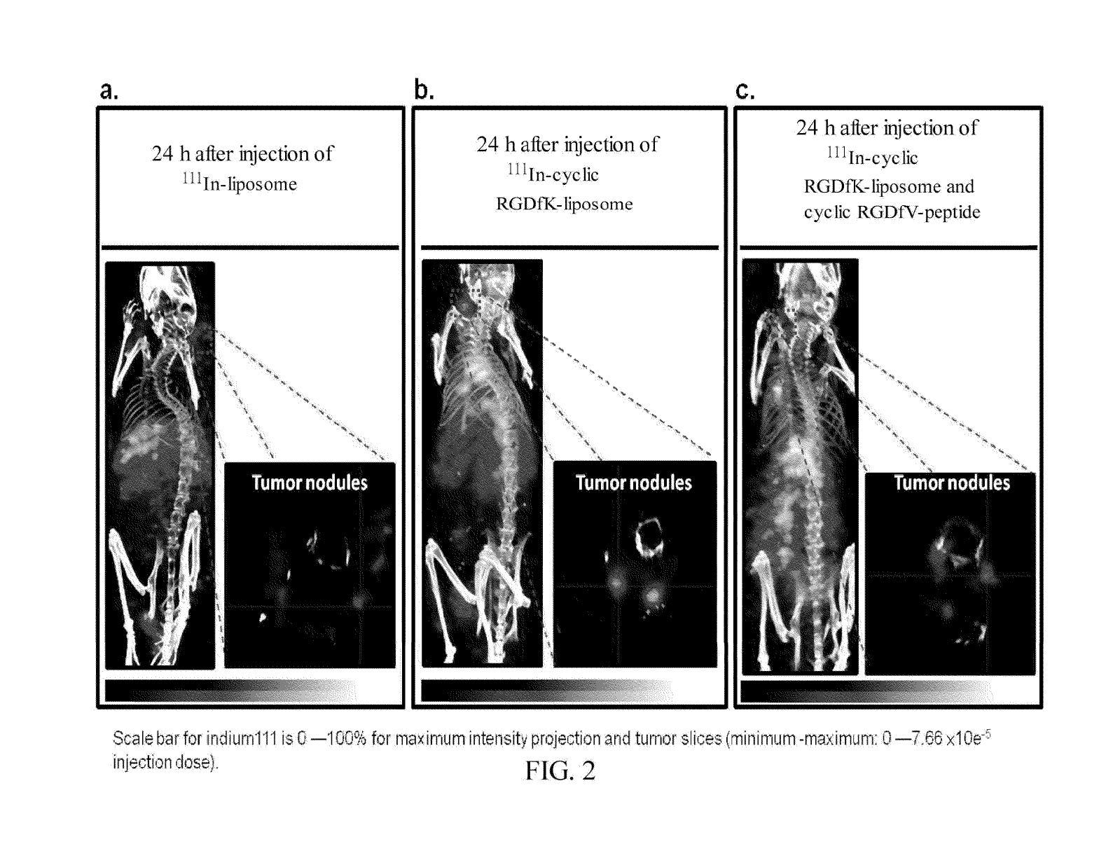

Referring to FIG. 2, compared with mice receiving ¹¹¹In labeled liposome (referred to as ¹¹¹In-liposome for short hereafter), due to accumulation of the ¹¹¹In labeled cyclic RGDfK-liposome (referred to as ¹¹¹In-cyclic RGDfK-liposome for short hereafter), the mice receiving the ¹¹¹In-cyclic RGDfK-liposome exhibit clear tumor nodule nuclear images.

Furthermore, the specificity of the ¹¹¹In-cyclic RGDfK-liposome serving as a target at the tumor site can be confirmed with mice that are jointly injected with the ¹¹¹In-cyclic RGDfK-liposome and the cyclic RGDfV peptide. The data shows that compared with mice without receiving the cyclic RGDfV peptide, radioactive signals in tumors of mice receiving the cyclic RGDfV peptide in picture b and picture c of FIG. 2 are actually reduced. Furthermore, it can be found through comparing picture a with picture b and picture c of FIG. 2 that the accumulation in tumor injected with the cyclic RGDfV peptide is less than that in tumor merely administered with ¹¹¹In-cyclic RGDfK-liposome, indicating that accumulation in tumor is actually caused by the cyclic RGDfK.

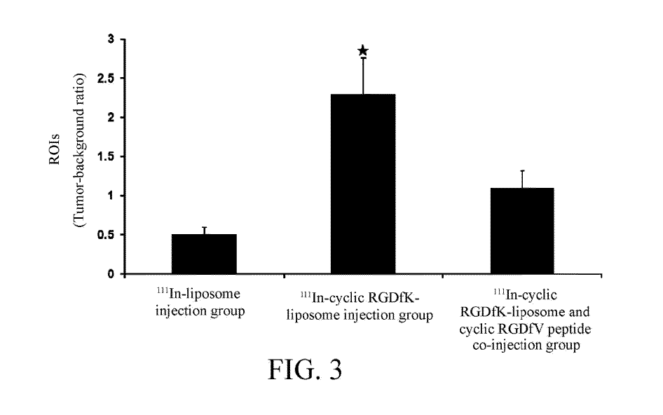

Furthermore, referring to FIG. 3, in the region of interest (ROI) of the tumor tissue, compared with the ¹¹¹In-liposome, the ¹¹¹In-cyclic RGDfK liposome has a higher tumor-to-background ratio, and data has significant difference. Therefore, besides the ¹¹¹In-cyclic RGDfK liposome can identify the tumor by means of the cyclic RGDfK peptide, the position of tumor also has the effect of promoting accumulation of the ¹¹¹In-cyclic RGDfK liposome.

Referring to Table 1 below, the tissue absorption of the two radioactive liposomes, especially the absorption in the tumor and blood, is analyzed, and the absorption of the ¹¹¹In-cyclic RGDfK liposome in the tumor and blood is respectively 5.3% ID/g and 1.1% ID/g, while the absorption of the ¹¹¹In-liposome in the tumor and blood is respectively 2.2% ID/g and 2.1% ID/g. The tumor-to-blood ratios of the two radioactive liposomes are compared, the tumor-to-blood ratio in the mice receiving the ¹¹¹In-liposome is 1.04, while the tumor-to-blood ratio in the mice receiving the ¹¹¹In-cyclic RGDfK-liposome is significantly increased to 4.8.

TABLE 1
Dose ratio of the ¹¹¹In-cyclic RGDfK liposome and the ¹¹¹In-
liposome in tissue samples, and the tumor-to-blood ratio
in mice without tumor metastasis of human melanoma cells

| | Tumor | Blood | Tumor-to- |
|---|---|---|---|
| | (% ID/g) | (% ID/g) | Background Ratio |
| ¹¹¹In-cyclic RGDfK liposome | 5.3 ± 0.09 | 1.0 ± 0.1 | 4.8 |
| ¹¹¹In-liposome | 2.2 ± 0.07 | 2.1 ± 0.02 | 1.04 |

### (II) Test Example 2

### ¹¹¹In-Cyclic RGDfK-Liposome in Nano SPECT/CT Imaging of Animal Model with Spontaneous Micrometastases of Human Melanoma Cells

1. Experimental Methods

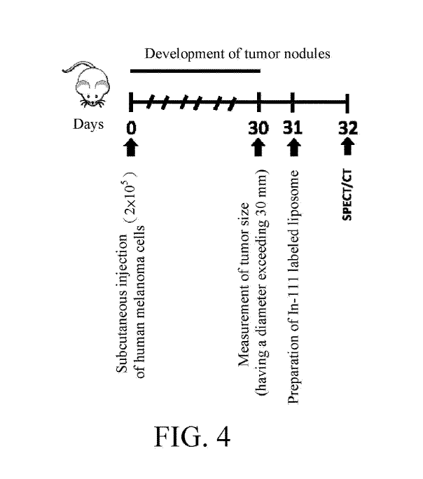

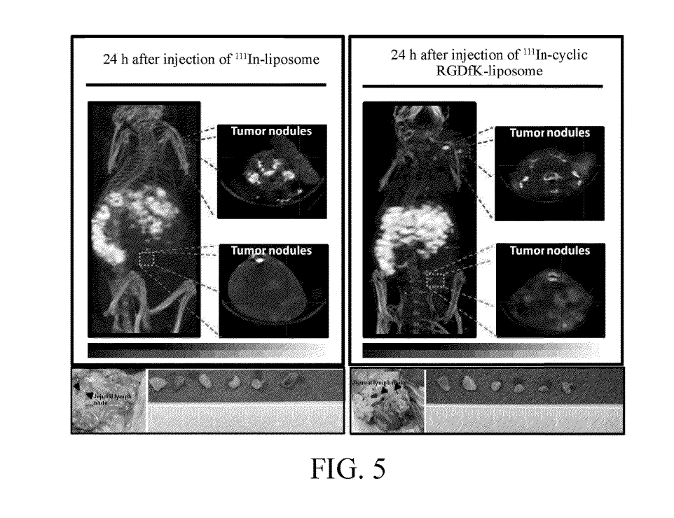

The animal used in this test example were female BALB/c AnN. Cg-Foxn1^{nu}/Cr1Nar1 mice, and 2×10⁵ A375.S2 human melanoma cells were injected into a nude mouse at the neck. 30 days after injection, the animal had developed a nodule having a diameter of about 30 mm. In order to determine the in vivo distribution of the radioactive liposome at 24 h after injection by using Nano SPECT/CT®^{plus}, first, the mouse was injected with ¹¹¹In labeled liposome (50 uCi) and anesthetized with 1.5% isoflurane at 24 h after injection, and nuclear images of the mouse were captured. For the experimental procedure, reference can be made to FIG. 4.

2. Experimental Results

Referring to FIG. 5, in this test example, another animal model with xenotransplantation of human melanoma cells was used to evaluate the efficiency of the ¹¹¹In-cyclic RGDfK liposome serving as the target at tumor site. FIG. 5 shows that in the mice injected with the ¹¹¹In-liposome, micrometastases can be found at mesenteric lymph nodes. By comparison, referring to the bottoms of picture a and picture b in FIG. 5 and Table 2 below, enlargement mesenteric lymph nodes were picked out, and the radioactivity of ¹¹¹In was analyzed. It is found that the absorption of the ¹¹¹In-cyclic RGDfK-liposome in the tumor and the blood of the mice is respectively 6.2% ID/g and 1.1% ID/g, it can be further confirmed that due to the accumulation of the ¹¹¹In-cyclic RGDfK-liposome, the mice injected with the ¹¹¹In-cyclic RGDfK-liposome exhibit clear tumor nuclear images.

On the other hand, the radioactivities in the tumor and the blood of the mice injected with ¹¹¹In-liposome are 2.9% ID/g and 2.1% ID/g.

TABLE 2
Dose ratio of the ¹¹¹In-cyclic RGDfK liposome and the ¹¹¹In-
liposome in tissue samples, and the tumor-to-blood ratio in
mice with spontaneous micrometastases of human melanoma cells

| | Tumor | Blood | Tumor/Blood |
|---|---|---|---|
| | (% ID/g) | (% ID/g) | Ratio |
| ¹¹¹In-cyclic RGDfK liposome | 6.2 ± 0.12 | 1.1 ± 0.05 | 5.6 |
| ¹¹¹In-liposome | 2.9 ± 0.09 | 2.1 ± 0.1 | 1.3 |

(III) Test Example 3: Expression of Cell αᵥβ₃ Integrin in Mesenteric Lymph Nodes

1. Experimental Methods

Cells (1.10⁶/ml) isolated from the mesenteric lymph nodes were cultured with 1 μg FITC-labeled mouse anti-human CD51/CD61 or FITC-labeled mouse IgG1κ isotype control monoclonal antibody for 60 min at 4° C., and then analyzed by using a flow cytometer.

2. Experimental Results

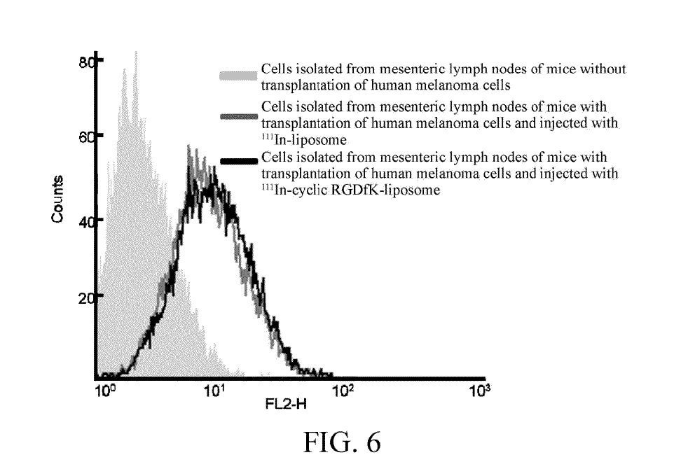

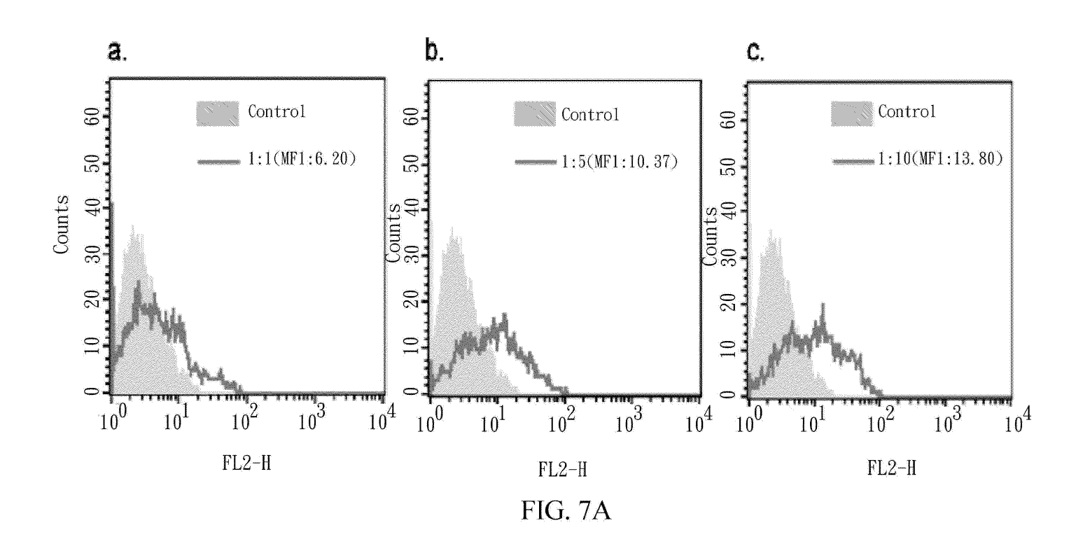

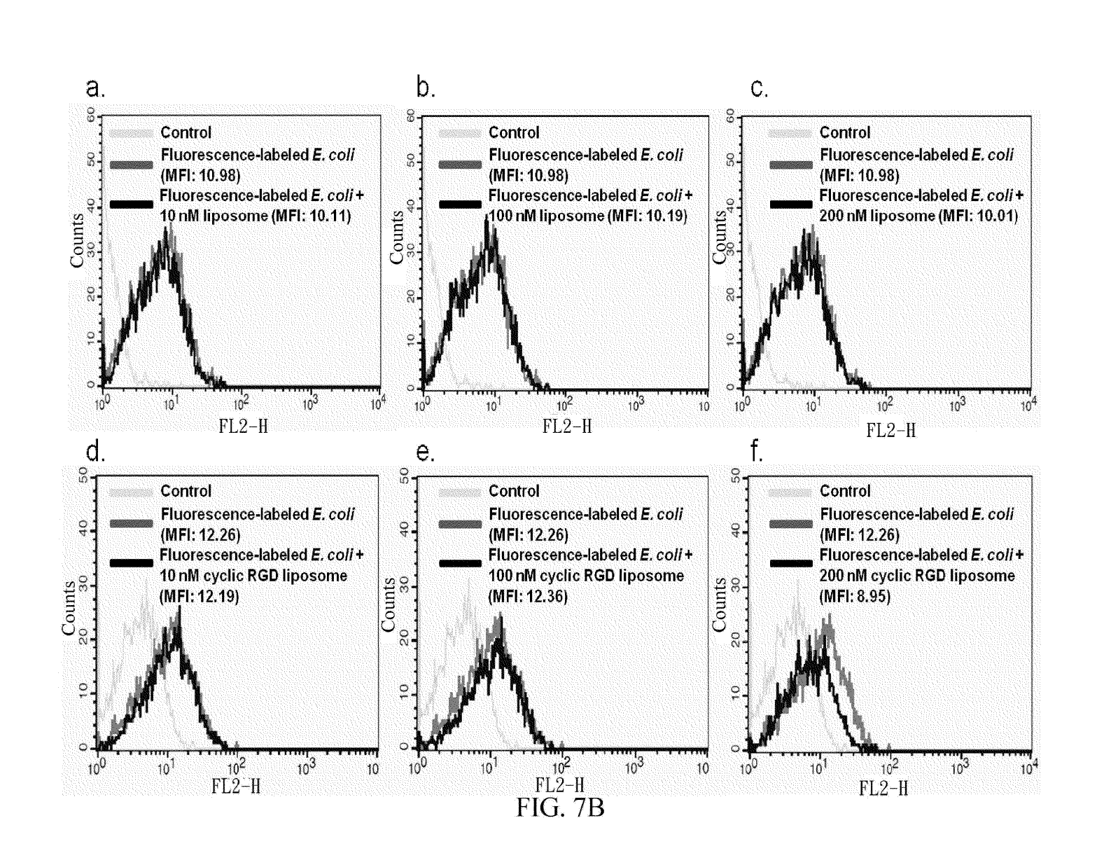

As shown in FIG. 6, the cell composition of tumor nodules picked out from the mesenteric lymph nodes is verified by FITC mouse anti-human CD51/CD61 monoclonal antibody staining performed on the cells. The cells isolated from the tumor actually express the αᵥβ₃ integrin, and referring to Table 2 above and compared with the tumor-to-background ratio (1.3) of the mice receiving the ¹¹¹In-liposome, the tumor-to-background ratio of the mice receiving the ¹¹¹In-cyclic RGDfK-liposome is significantly increased to 5.6.

### (IV) Test Example 4

### Influence of Cyclic RGDfK-Liposome on Functions of Phagocytes

1. Experimental Methods

(1) Phagocytosis Test

Phagocytosis of fluorescence-labeled Escherichia coli BioParticles was performed according to the experimental plan of the manufacture. Immune cell RAW 264.7 and fluorescence-labeled Escherichia coli BioParticles (at a ratio of 1:10) were reacted in Dulbecco's modified Eagle's medium containing 10% hot decomplemented fetal bovine serum for 1 h at 37° C. Tryphan blue was added to quench the fluorescence of particles that are not swallowed, and then the test sample was analyzed by using a flow cytometer.

(2) Test of Generation of Reactive Oxygen Species

In the case of the presence or absence of the liposome or the cyclic RGDfK modified liposome, RAW 264.7 cells (1×10⁶ cells/ml) and 100 μg dichlorodihydrofluorescein diacetate were reacted in 0.1 ml sterile phosphate buffer for 1 h at 37° C. For the lipopolysaccharide-stimulated reactive oxygen species generation group, in the case of the presence or absence of liposome or the cyclic RGDfK modified liposome, RAW 264.7 cells (1×10⁶ cells/ml), 200 μg lipopolysaccharide and 100 μg dichlorodihydrofluorescein diacetate were reacted in 0.1 ml sterile phosphate buffer for 1 h at 37° C. These test samples were analyzed by using a flow cytometer.

2. Experimental Results

Whether the cyclic RGDfK-liposome has influence on the phagocytic activity of mouse macrophages RAW 264.7 is tested in the following test examples.

Referring to figure A of FIG. 7, the proportion of cells to fluorescence-labeled bacterial particles is determined by using a flow cytometer-based system, and it is found that the proportion 1:10 is the optimal condition for phagocytosis test.

Additionally, referring to figure B of FIG. 7, in the case of the presence of the cyclic RGDfK-liposome (10 or 100 nM), it can be found that high-concentration cyclic RGDfK-liposome will slightly decrease the phagocytic ability of cells, compared with the groups merely treated with the liposome (referring to picture a to picture c of B).

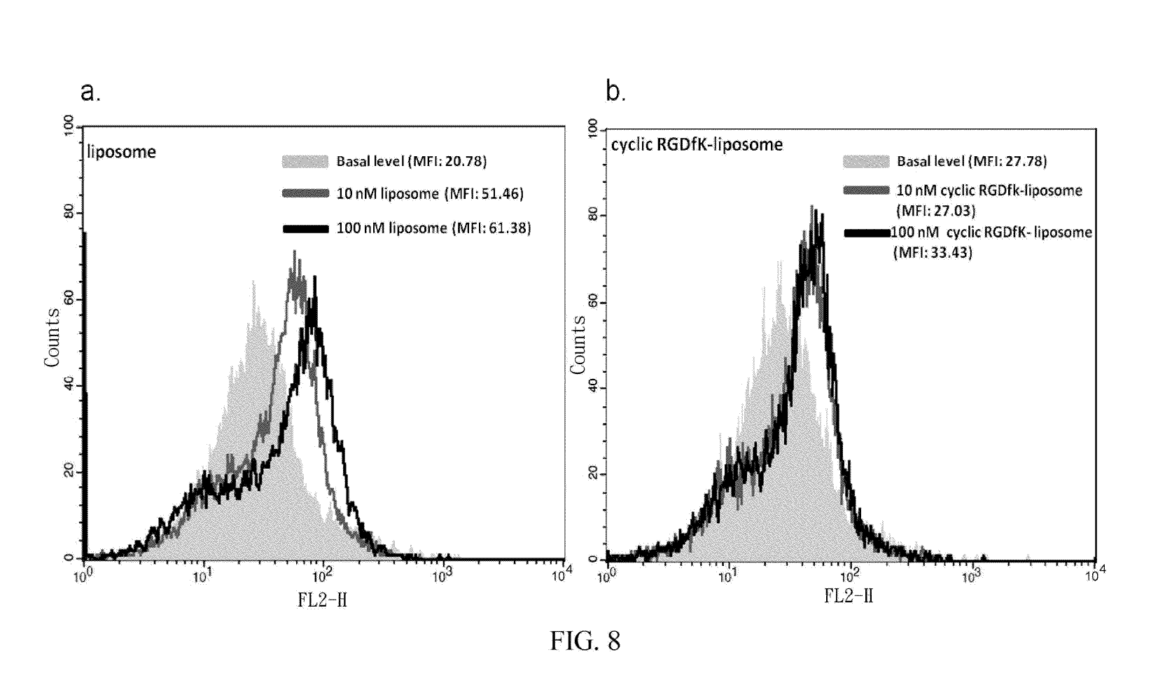

Since phagocytosis accompanies with the generation of reactive oxygen species, whether the cyclic RGDfK-liposome has influence on functions of phagocytes, especially on the reactive oxygen species generation action of lipopolysaccharide that is known simulating reactive oxygen species generation, can be determined by detecting the generation of reactive oxygen species. Picture a and picture b in FIG. 8 show the influence of the groups treated with the cyclic RGDfK-liposome and the liposome in the case of the absence of lipopolysaccharide.

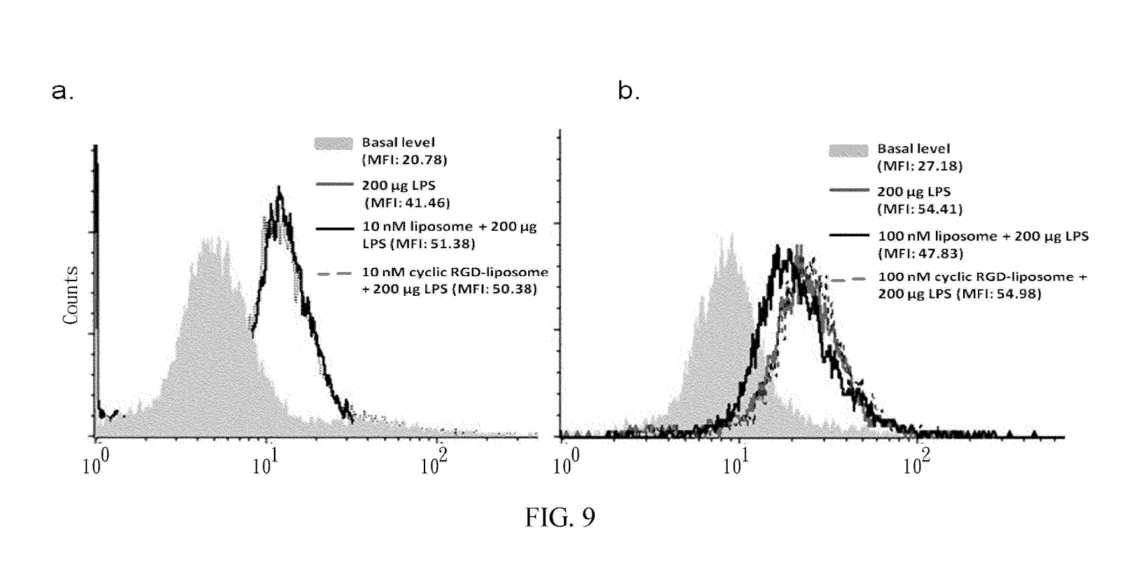

By comparison, picture b in FIG. 9 shows that the liposome inhibits the capability of phagocyte of generating reactive oxygen species, but the cyclic RGDfK-liposome of different concentrations will not inhibit the capability of generating reactive oxygen species stimulated by lipopolysaccharide.

These experimental results verify that cyclic RGDfK-modified targeting liposome has no significant influence on normal functions of immune phagocytes, that is, the results show that although the cyclic RGDfK liposome has influence on phagocytosis and reactive oxygen species generation of the immune cells, but the influence is not significant and can be ignored.

In view of the above, in the embodiments of the present disclosure, by means of the characteristic of high expression of the αᵥβ₃ integrin in tumor tissues in extracellular matrix, the αᵥβ₃ integrin is used as the target receptor. Furthermore, since the cyclic RGD peptide with fixed configuration can be directly used as the reagent or nanoparticles for drug delivery, and can be effectively delivered to tumor vessels with high expression of αᵥβ₃ integrin, the pharmaceutical composition for detecting human melanoma cells with the cyclic RGDfK-modified liposome as radioisotope carrier of the present disclosure has the following advantages:

1. Strong and specific action: By means of specific binding of the gamete and the receptor, the action between the liposome and the target cell is increased, and poor endocytosis and endosomal escape are improved.

2. Identifying cancers with micrometastases: The integrin plays an important role in cell growth and metastasis, so by means of identification of the cyclic RGDfK and the αᵥβ₃ integrin, cancer cells with metastasis can be monitored.

3. No immunogenicity: In the immune cells in vitro endocytosis and reactive oxygen species generation tests, the influence of cyclic RGDfK-liposome on the immune system may be not considered.

4. High detection sensitivity: Radioisotope ¹¹¹In is a γ radiation source and has the maximum energy of 245 keV, and is applicable in γ-development and tracers.

Therefore, compared with the ¹¹¹In-liposome, the ¹¹¹In-cyclic RDGfK-liposome has better Nano SPECT/CT images, and has considerable potential in applications of melanoma diagnosis and tumor screening, lymphatic metastasis detection and post-surgical monitoring.

Those skilled in the art should understand that, without departing from the spirit of the present invention, various variations can be made according to the implementation aspects of the present invention. Therefore, it is obvious that the illustrated implementation aspects are not used for limiting the present invention, but are intended to encompass modifications made in the spirit and scope of the present invention under the definition of the following claim.

1. A pharmaceutical composition for detecting human melanoma cells, comprising:
a liposome, having an outer membrane;
a biomolecule, connected to the outer membrane of the liposome, and having specificity for αᵥβ₃ integrin; and
a radionuclide, selected from the group consisting of indium, iodine, rhenium, gallium-67, gallium-68 and technetium.

2. The pharmaceutical composition according to claim 1, wherein the liposome is further modified by polyethylene glycol.

3. The pharmaceutical composition according to claim 1, wherein the biomolecule is a cyclic peptide.

4. The pharmaceutical composition according to claim 3, wherein the cyclic peptide is cyclic RGDfK.

5. The pharmaceutical composition according to claim 1, wherein the radionuclide is indium-111.

6. A method for detecting human melanoma cells, comprising:
a. administering the pharmaceutical composition according to claim 1 to a subject having human melanoma cells, wherein the human melanoma cells comprise αᵥβ₃ integrin; and
b. detecting data of specific binding of a biomolecule of the pharmaceutical composition and the αᵥβ₃ integrin, so as to detect the transfer degree of the melanoma cells.

7. The method according to claim 6, wherein the human melanoma cells are A375.S2.

8. The method according to claim 6, wherein the data of specific binding is determined by using nano single ingle photon emission computed tomography (SPECT/CT) images.

9. The method according to claim 6, wherein the subject is an animal with xenotransplantation.

10. A kit for detecting human melanoma cells, comprising:
the pharmaceutical composition according to claim 1; and
an operating instruction, wherein the operating instruction comprises:
a. administering the pharmaceutical composition to a subject having human melanoma cells, wherein the human melanoma cells comprise αᵥβ₃ integrin; and
b. detecting data of specific binding of a biomolecule of the pharmaceutical composition and the αᵥβ₃ integrin, so as to detect the transfer degree of the melanoma cells.

11. The kit according to claim 10, wherein the human melanoma cells are A375.S2.

12. The kit according to claim 10, wherein the data of specific binding is determined by using nano single ingle photon emission computed tomography (SPECT/CT) images.

13. The kit according to claim 10, wherein the subject is an animal with xenotransplantation.
